(19) United States
(12) Patent Application Publication
(10) Pub. No.: US 2026/0105213 A1
(43) Pub. Date: Apr. 16, 2026

(54) SYSTEM AND METHOD FOR GENERATING AN APPLIANCE

(71) Applicants: Aadhav Sundar, Peachtree City, GA (US); Cole Digirolamo, Lisle, IL (US); Daniel Lee, Duluth, GA (US)

(72) Inventors: Aadhav Sundar, Peachtree City, GA (US); Cole Digirolamo, Lisle, IL (US); Daniel Lee, Duluth, GA (US)

(73) Assignee: Stride Labs, Peachtree City, GA (US)

(21) Appl. No.: 18/914,343

(22) Filed: Oct. 14, 2024

(51) Int. Cl. G06F 30/27; A61B 5/00; A61B 5/107; A61F 5/14; B33Y 50/00; B33Y 80/00; G06F 3/08

(52) U.S. Cl. CPC G06F 30/27; A61B 5/0064; A61B 5/1077; A61B 5/702; A61F 5/14; G06F 3/08; B33Y 50/00; B33Y 80/00

(57) ABSTRACT

In some embodiments systems and methods are provided for an automated system for generating an appliance for a body part that utilizes artificial intelligence and machine learning to identify points in a scan, generate a surface from the identified points, and generate the appliance for the body part.

### BACKGROUND

### Field of the Invention

The present disclosure is related to a system and method for generating an appliance for a body part.

### Description of the Related Art

There are four typical methods for generating an orthotic appliance for a foot, plaster, foam box and direct mold. All methods require certified, highly skilled professionals at different points in the process that results in an expensive, labor-intensive process that requires weeks to months to fulfill a patient's order. The current labor-intensive methods are also error prone with a high rate of return for corrections and adjustments to be performed by the trained/certified technician.

The four methods are briefly described here for context. The plaster method begins with a thorough assessment of the patient's foot structure, gait, and specific needs by a certified/trained professional. The patient's foot is cleaned and dried to ensure no debris or moisture interferes with the casting. The patient is either seated or lying down with their foot positioned correctly (usually in a neutral or subtalar neutral position). Plaster bandages are soaked in water and wrapped around the foot and ankle. The plaster is molded to capture the foot's shape and any necessary corrections. The plaster is allowed to harden, usually taking 5-10 minutes. Once set, the plaster cast is carefully cut and removed from the foot. The negative plaster cast is filled with liquid plaster or a similar material to create a positive model of the foot. Adjustments are made to the positive model to correct any deformities or to add specific corrections. Layers of thermoplastic, leather, or other materials are heated and molded over the positive model. The orthotic is trimmed, smoothed, and finished to fit comfortably in the patient's shoe.

The foam box method is similar to the plaster cast method, an initial assessment of the patient's foot is conducted by trained/certified professional. A special foam impression box is used, designed to capture the foot's contour. The patient stands or sits with their foot over the foam box. The foot is pressed into the foam to create an impression, capturing the foot's shape and any deformities. The foam impression is sent to a lab where it is used to create a positive mold, typically using plaster. Similar to the plaster cast method, the positive mold is modified, and layers of material are applied to create the orthotic. The orthotic is trimmed, smoothed, and finished by a trained professional before it is supplied to the patient.

The direct mold method is similar to the plaster method and the foam box method. The patient is assessed by a trained/certified professional to determine the foot's structure and needs. Thermoplastic or other moldable materials are prepared and heated. The patient's foot is positioned correctly, often in a neutral position. The heated material is directly applied to the bottom of the foot or inside a slipper cast. It is molded and pressed to take the shape of the foot. The material is allowed to cool and harden while maintaining the foot's contour. A trained/certified technician makes any necessary adjustments and corrections to the molded material and then the orthotic is trimmed and smoothed for comfort.

### SUMMARY

In some embodiments of the present disclosure systems and methods are provided for an automated system for generating an appliance for a body part that utilizes artificial intelligence and machine learning to identify key points in a scan, generate a surface from the identified points, and send that generated surface to a manufacturing system to generate the appliance for the body part.

The systems and methods reduce the cost of generating a fitted device by at least 10 times by automating the process of modeling utilizing artificial intelligence and machine learning models to model the body part. The current methods used in the marketplace require certified/trained technicians at different times in the process to evaluate, make corrections or adjustments and finalize the product before it is ready for the customer, these processes are time-intensive and the costs are prohibitively expensive.

The systems and methods also reduce the error rate of modeling to less than 1% compared to current methods. Current methods have a significantly high return rate of greater than 10%.

### BRIEF DESCRIPTION OF THE DRA WINGS

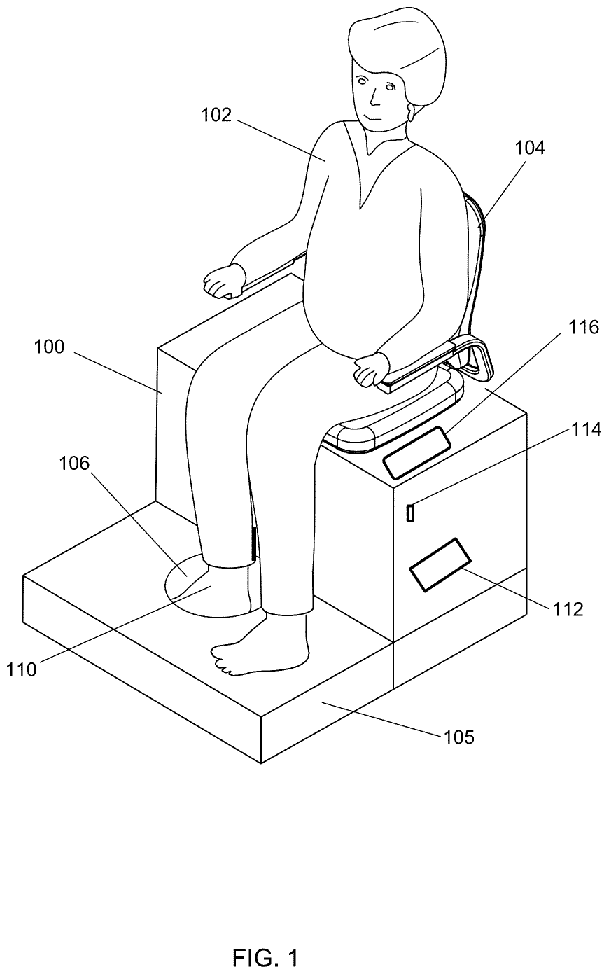

FIG. 1 is a perspective view of the system showing a user in a seated position with a foot positioned inside the device according to one embodiment of the disclosure.

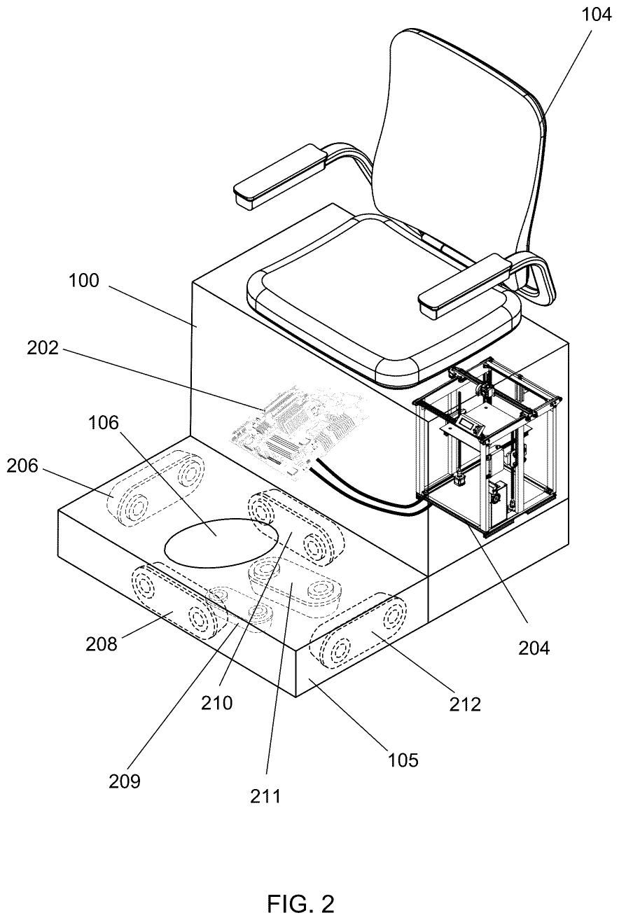

FIG. 2 is a perspective view of the internal components according to one embodiment of the disclosure.

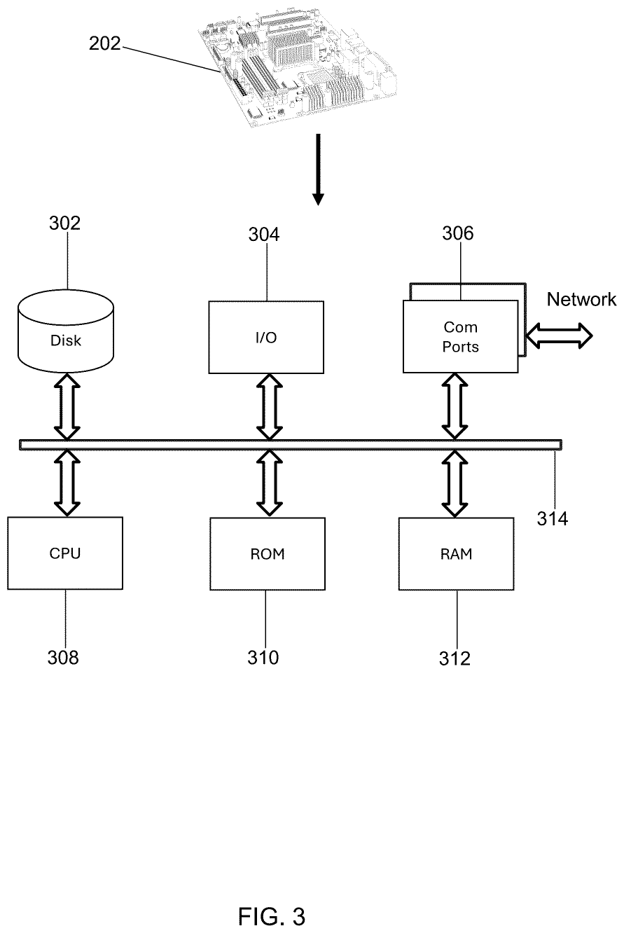

FIG. 3 is a perspective view of a computing device according to one embodiment of the disclosure.

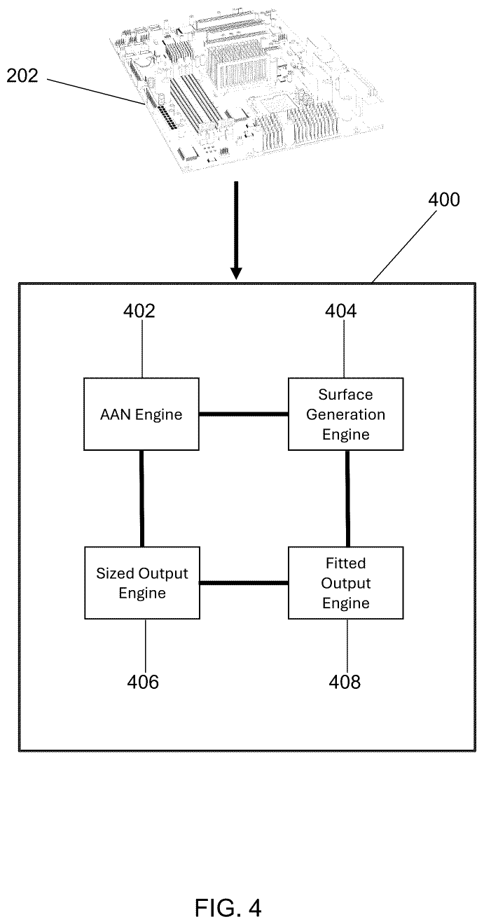

FIG. 4 is a diagram of computing modules according to one embodiment of the system.

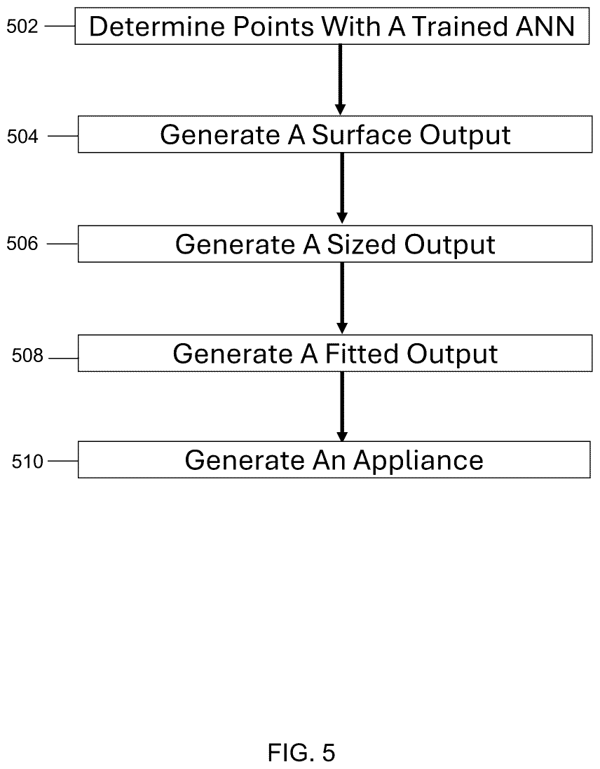

FIG. 5 is a flowchart illustrating a method of generating an appliance according to one embodiment of the disclosure.

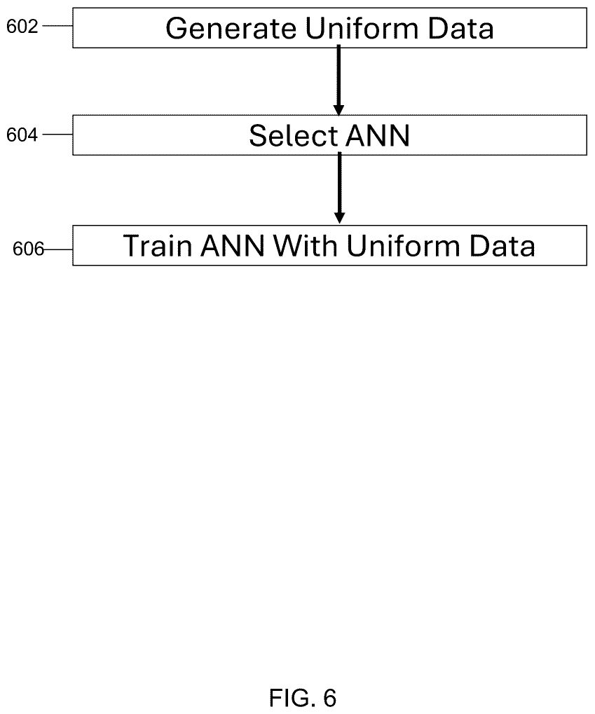

FIG. 6 is a flowchart illustrating a method of training an artificial intelligence algorithm according to one embodiment of the disclosure.

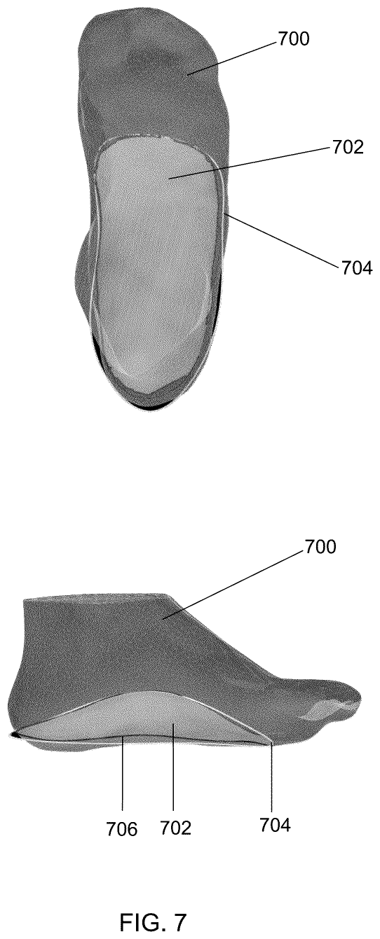

FIG. 7 is two perspective views 7A & 7B of a 3D scan of a user's foot.

### DETAILED DESCRIPTION

In the following description of embodiments, reference is made to the accompanying drawings which form a part hereof, and in which it is shown by way of illustration specific embodiments that are optionally practiced. It is to be understood that other embodiments are optionally used and structural changes are optionally made without departing from the scope of the disclosed embodiments.

The embodiments of this description provide at least the following benefits and improvements over current methods of generating an appliance with reference to the generation of an orthotic device. However, the embodiments of the description are not limited as such, and may be applied to any system and/or method of generating an appliance, such as for example a dental appliance, brace, cast, or a fitted device for a body part, or any similar appliance intended to be fitted to a body part.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

FIG. 1 is a perspective view of the system showing a user in a seated position with a foot positioned inside the device according to one embodiment of the disclosure. FIG. 1 shows an embodiment of an appliance generator 100. In some embodiments, the appliance generator 100 may have a seat 104 to provide a seating position for a user 102. In some embodiments the appliance generator 100 may be configured scanning portion 105 having an opening 106 for inserting a foot 110 of the user 102 into the scanning portion 105. In some embodiments, the seat 104 provides a stable seating position for the user 102 to insert foot 110 into the opening 106 of the scanning portion 105 for scanning inside the appliance generator 100. The appliance generator 100 may be configured with a port 112 in which an appliance may be deposited after the appliance generator 100 has generated the appliance. In some embodiments, the user 102 may retrieve the appliance from the port 112.

In some embodiments the appliance generator 100 may be configured without a seat, and the user 102 may stand while the scanning portion 105 scans. In some embodiments the appliance generator 100 may be configure to support the entire weight of the user 102, such as a support member for supporting the user 102 in a prone position or in a suspended position. In some embodiments, the scanning portion 105 may be configured in a position in which the foot 110 of user 102 may be scanned.

In some embodiments the appliance generator 100 of FIG. 1 may be configured without the port 112. In some embodiments the appliance generator 100 may scan the foot 110 of the user 102 and send the data to an external system for generating the appliance. In some embodiments the appliance generator 100 may be configured to receive a scan of the foot 110 of user 102 from an external source, for example a scan may be generated by a third party such as a physician or technician and sent via an electronic method to the appliance generator 100 and the appliance may be generated for the user 102 without scanning the foot 110 of the user 102. In some embodiments the appliance generator 100 may be configured with an external port 114 for receiving and/or outputting scan information of a user 102. In some embodiments the appliance generator 100 may be configured with a device for receiving scan data of a user 102 wirelessly.

In some embodiments the appliance generator 100 of FIG. 1 may be configured with a user interface 116. In some embodiments the user interface 116 provides an interface for the user 102 to interact with the systems of the appliance generator 100. In some embodiments the user interface 116 may be configured to receive user information such as for example, height, weight, and other data that is related to a condition of the user 102.

In some embodiments, the user 102 may input information via user interface 116 and/or the appliance generator 100 may be capable of receiving electronically from a wired or wireless connection information related to a user 102 such as a diabetic condition, blood disorders, problems with joints (such as arthritis), bone deformities, circulation issues (such as peripheral vascular disease), nerve, muscle, brain, spinal cord, or inner ear diseases or injuries, arthritis of the hips, knees, ankles, or feet, cerebellar disorders affecting coordination and balance, parkinson's disease, multiple sclerosis, stroke, cerebral palsy, hemiplegia, spinal stenosis, herniated disk, and other underlying conditions.

FIG. 2 is a perspective section view of internal components according to one embodiment of the disclosure. In some embodiments the appliance generator 100 may be configured with a computing device 202 for carrying out the processing of the appliance generator 100. The computing device 202 includes at least one processing unit (CPU) in communication with a mass memory via a bus. The computing device 202 also includes one or more network interfaces for electronic communication via wired or wireless communication, an audio interface, an input/output interface, a haptic interface, an optional global positioning systems (GPS) receiver and a camera(s) or other optical, thermal, or electromagnetic sensors The computing device 202 may be configured with input/output interface for communicating with external devices, using communication technologies, such as USB, infrared, Bluetooth®, or the like. The computing device 202 can include one camera/sensor, or a plurality of cameras/sensors, and may also be configured with a display, a keypad, and an illuminator, as understood by those of skill in the art. The positioning of the camera(s)/sensor(s) on the device may change per model of appliance generator 100 capabilities, and the like, or some combination thereof.

In some embodiments the computing device 202 may be configured with an application specific integrated circuit (AISC) or a field-programmable gate array (FPGA). Alternatively, or in combination, the functions and operations can be implemented using special purpose circuitry (e.g., logic circuitry), with or without software instructions. Embodiments can be implemented using hardwired circuitry without software instructions, or in combination with software instructions. Thus, the techniques are not limited to any specific combination of hardware circuitry and software, nor to any particular computing source for the instructions executed by a computing device.

In some embodiments, the computing device 202 is a system including one or more processors. Examples of the processing device can include a microcontroller, a central processing unit (CPU), special purpose logic circuitry (e.g., an FPGA, an ASIC, etc.), a system on a chip (SoC), a programmable logic controller (PLC) or another suitable processor. In some embodiments the computing device 202 may be configured as a quantum computer or any similar computing system or any combination thereof.

In some embodiments, the computing device 202 may be configured to, through the input output interface, calibrate and initiate scan hardware 206, scan hardware 208, scan hardware 209, scan hardware 210, scan hardware 211, and scan hardware 212. In some embodiments, User 102 may place a foot 110 inside opening 106 and computing device 202 may scan with scan hardware 206 scan hardware 208, scan hardware 209, scan hardware 210, scan hardware 211, and scan hardware 212, the foot 110 of the user 102. Computing device 202 may be configured to process scan data, and send scan data to an appliance generation device 204. In some embodiments, scan hardware 206, scan hardware 208, scan hardware 209 scan hardware 210, scan hardware 211 and scan hardware 212 may be configured as 3D cameras positioned on the interior surfaces of scanning portion 105. The number of 3D cameras inside the scanning portion 105 is not limited to the number illustrated in FIG. 2 and may include more 3D cameras or fewer 3D cameras.

In some embodiments scanning portion 105 of the appliance generator 100 may be configured with a scanning system to obtain a scan or a 3D image for analysis. The scanning portion 105 may utilize a laser or a combination of lasers, infrared light, single photon, an active (artificial light) or passive imaging system, or any combination thereof. In some embodiments the scanning portion 105 may be configured to utilize stereo optical and/or stereo vision, time of flight (ToF), structured light 3D imaging, LIDAR, mid-infrared single photon systems or any combination thereof to scan a body part. In some embodiments the scanning portion 105 may utilize any frequency of visible and non-visible light to scan a body part such as for example computerized tomography (CT) x-ray backscatter formally known as Compton scattered radiation which utilizes diffusely reflected x-rays subsequent to reflected collimated x-rays impacting the target, additionally techniques such as x-ray photoelectron spectroscopy and/or x-ray surface analysis techniques may be used to scan a body part. In some embodiments, the scanning portion 105 may utilize mechanical surface detection techniques to scan a body part such as for example mechanical surface deflection techniques that utilize mass/weight measurement, and/or techniques that utilize sensor deflection such as piezoelectric deflection or any combination thereof. In some embodiments the scanning portion 105 may utilize acoustic or ultrasonic scanning techniques to scan a body part such as for example ultrasound, and/or adaptive pulse ultrasound excitation.

In some embodiments, continuing with reference to FIG. 2, the appliance generator 100 may be configured with an appliance generation device 204 for generating an appliance for a body part. In some embodiments the computing device 202 of the appliance generator 100 may scan with scan hardware 206, scan hardware 208, scan hardware 209, scan hardware 210, scan hardware 211, scan hardware 212, the body part of a user 102, the computing device 202 may be configured to then process the scan with a series of techniques (explained in detail below) to produce a manufacturable model of body part of the user 102, the computing device 202 may be configured to then send the manufacturable model to the appliance generation device 204 for manufacture.

In some embodiments, the appliance generation device 204 may be configured as an additive device, for example a printing technology that utilizes directed energy deposition, material extrusion, sheet lamination and/or material deposition techniques or any combination thereof including combining additive and subtractive manufacturing techniques.

In some embodiments the appliance generation device 204 may be configured as a subtractive device, examples of such may include machining, CNC machining, electrical discharge machining, laser cutting, and/or water jet cutting and/or any combination thereof for generating a manufacturable model of the body part of the user 102 to generate an appliance. The above-described subtractive manufacturing techniques may be used individually or in combination, those skilled in the art will recognize that the subtractive manufacturing techniques may additionally be utilized in combination with the additive manufacturing techniques described to generate an appliance for a body part.

FIG. 3 is a perspective view of a computing device 202 according to one embodiment of the disclosure. In some embodiments, the computing device 202 may be configured as a system connected by bus 314, the system including one or more processors CPU 308, a long-term storage device such as disk 302, an input output interface I/O 304, a read only memory device ROM 310, random access memory RAM 312 and communication port 306. The computing device 202 configured to store one or more programs for carrying instructions of scanning a body part, analyzing a scan of a body part and determining a manufacturable model of an appliance on the CPU 308, the one or more programs stored in one of disk 302, ROM 310, RAM 312. In some embodiments, the computing device 202 may be configured to access one more programs stored externally through communication ports 306, retrieving the one or more programs from a cloud network and/or a private storage network. In some embodiments the one or more programs may be stored in one or more modules on one of disk 302, ROM 310, RAM 312.

FIG. 4 is a diagram of computing modules according to one embodiment of the system. In some embodiments the computing device 202 may be configured with one or more modules for executing the one or more programs for generating an appliance. In some embodiments the module 400 may be stored and executed on an external network, such as cloud storage, and/or may be accessed by endpoint API. In some embodiments the computing device 202 may be configured with a Module 400 having one or more artificial intelligence engines AAN engine 402. In some embodiment the ANN engine 402 may be configured to determine with a trained artificial neural network algorithm at least one point from data related to a body part. In some embodiments the ANN engine 402 may be configured to determine a disease state from a scan. For example certain disease states such as diabetes have predictable features that appear in scans that may be used to train the ANN engine 402. In some embodiments the ANN engine 402 may be configured to execute one or more artificial neural network architecture's stored either locally on disk 302 or remotely on cloud storage or a private storage system. The ANN engine 402 may be configure to execute any one of a feed forward recursive neural network algorithm, a generative adversarial algorithm (GAN), naïve bayes classifier, decision tree, logistic regression, random forest, support vector machine, k-nearest neighbor, linear regression, lasso, k-mean clustering, multivariate regression, multiple regression algorithm, fuzzy c-means algorithm, expectation maximization algorithm, hierarchical clustering algorithm or any combination thereof.

In some embodiments the module 400 may be configured with surface generation engine 404. In some embodiments the surface generation engine 404 may be configured to access one or more surface generation algorithms and generate a surface with an outer boundary from the at least one point determined by the AAN engine 402. In some embodiments, the generated surface may be visualized as a mesh point cloud having XYZ components. In some embodiments the surface generation engine 404 may be configured to generate a surface from the at least one point utilizing one or more interpolation algorithms such as scattered point spline interpolation, linear algebraic algorithm, Laplacian method, numerical analysis, LaGrange polynomial and/or any similar technique or combination of techniques for determining a surface from the at least one point determined by the ANN engine 402.

In some embodiments the module 400 may be configured with a sized output engine 406. The sized output engine 406 may be configured to retrieve the surface and boundary generated by the surface generation engine 404, and retrieve scan data of the body part and determine a boundary optimization by comparing the surfaces of both data. In some embodiments the sized output engine 406 may determine the boundary optimization by utilizing one more algorithms such as for example ray tracing algorithm. The ray tracing algorithm casts a set of rays from the at least one point and boundary determined by the surface generation engine 404 toward and away from the center of the boundary to find the closest intersection with the scan data to generate a fitted output. In some embodiments the sized output engine 406 is not limited to using ray tracing to determine an optimization and may utilize other algorithms well known by those skilled in the art such as for example, Z buffer, scan line, list priority algorithm, reyes algorithm, and rasterization individually or in any combination thereof.

In some embodiments the module 400 may be configured with a fitted output engine 408. The fitted output engine 408 may be configured to extend data points determined by the sized output engine 406. In some embodiments the fitted output engine 408 may be configured to retrieve and/or receive data applicable to user 102 in order to determine the extension. In some embodiments, the extension determined by the fitted output engine 408 may be orthogonal to the plane of the surface and boundary determined by the sized output engine 406 and may add a thickness dimension to the data points. In some embodiments the fitted output engine 408 may utilize data from appliance manufacturer specific data, and/or user input data entered into user interface 116 and/or may be transferred over a network to computing device 202 and/or transferred over a network to module 400 or any combination thereof.

In some embodiments the user data input may be user preferences specific to the fitted output generated by the sized output engine 406, for example the user 102 may prefer a posting on the heel of the appliance, the fitted output engine 408 may be configured to utilize one or more algorithms to parametrically add vectors and faces to generate the posting. In some embodiments once the fitted output engine 408 has generated the posting the completed sized output may be sent by the computing device 202 to the appliance generation device 204 to be generated.

In some embodiments the fitted output engine 408 may be configured to utilize one or more algorithms to generate design changes to the fitted output according to manufacturer specific data for example in some instances the user 102 may prefer an appliance to be generated from a material manufactured by a third party supplier, which in this instance may include one or more manufacture specific data such as parametric design specifications, design alteration and/or configurations specific to the manufacturer. In some embodiments the fitted output engine 408 may be configured to utilize one or more algorithms to generate the design specifications in the sized output. The sized output including the design specifications may then be sent to the appliance generation device 204 for manufacture.

In some embodiments fited output engine 408 may be configured to generate a sized output utilizing one or more algorithms such as an interpolation algorithm, scattered point spline interpolation, linear algebraic algorithm, Laplacian method, numerical analysis, LaGrange polynomial and/or any similar technique or combination of techniques to generate a sized output.

FIG. 5 is a flowchart illustrating a method of generating an appliance according to one embodiment of the disclosure. The methods described below may be implemented on appliance generator 100 but are not limited as such and may be implemented on any computing system capable of carrying out the method, furthermore the method may be implemented in part or in some combination on the appliance generator 100. In some embodiments at Step 502 the system determines at least one point from data of a body part with a trained artificial intelligence network.

In some embodiments at Step 502 the method utilizes a trained artificial intelligence model (ANN) to determine at least one point from scan data of the user 102 foot 110. The at least one point represents anatomical landmarks on the foot 110 and the method may determine seven points or more from the regions of the foot 110. The anatomical landmarks are specific regions of the foot 110 utilized by one or more of the steps in the method for generating the appliance. The seven points may be determined by the ANN from a first metatarsal region, a fifth metatarsal region, a base of the 5^{th} metatarsal region, a navicular point region, a left heel point region, a right heel point region (i.e., lateral and medial regions), and a dorsal heel region of the foot 110. In some embodiments at Step 502 the method may determine additional points from the regions of the foot 110 such as for example a first metatarsal region, a third metatarsal region, a fifth metatarsal region, a navicular region, a mid-lateral arch region, a mid-heel region, a left heel region, a right heel region, and a top heel region.

In some embodiments at Step 504 the method may utilize one or more algorithms to generate a surface output utilizing the data from the determined points in Step 502. The determined points may be utilized to form the surface and boundary of the surface output. The method may utilize one or more algorithms such as interpolation for example a spline interpolation to create a smooth boundary curve along the determined points. The method at step 504 may utilize one or more algorithms such as scattered point spline interpolation, linear algebraic algorithm, Laplacian method, numerical analysis, LaGrange polynomial and/or any similar technique or combination of techniques for determining a surface output.

In some embodiments at Step 506 the method may utilize one or more algorithms to generate a sized output utilizing the data of the surface output generated in Step 504. The method at Step 506 may retrieve the surface and boundary generated at Step 504 and retrieve scan data of the foot 110 and determine a boundary optimization from the data of both surfaces. In some embodiments the method may determine the boundary optimization by utilizing one more algorithms such as for example ray tracing algorithm but is not limited to using ray tracing to determine an optimization and may utilize other algorithms well such as for example, Z buffer, scan line, list priority algorithm, reyes algorithm, and rasterization individually or in any combination thereof.

In some embodiments at Step 508 the method may utilize one or more algorithms to generate a fitted output from the data generated in the sized output of Step 506. The method may store and/or retrieve and/or receive data, the data may be appliance manufacturer specific data, and/or user data. The user 102 may configure certain parameters of the fitted output for example the user 102 may configure a posting on the heel of the appliance. The method may be configured to utilize one or more algorithms to parametrically add vectors and faces to the data points. The method may be configured to add a thickness to the data points by extending points along an orthogonal plane. The user 102 may configure parameters of the fitted output on a user interface 116 to change the shape of the appliance before it is manufactured by the appliance generation device 204.

In some embodiments at Step 508 the method may utilize one or more algorithms to generate design changes to the fitted output according to manufacturer specific data for example material specific data such as density of material or layering of materials, and adhesives. In some embodiments the manufacture specific data may alter the fitted output parametric design specifications, design alteration and/or configurations specific to the manufacturer. In some embodiments the method may utilize one or more algorithms to generate sized output based on the manufacturer specific design specifications.

In some embodiments at Step 508 the method may utilize one or more fitted algorithms such as an interpolation algorithm, scattered point spline interpolation, linear algebraic algorithm, Laplacian method, numerical analysis, LaGrange polynomial and/or any similar technique or combination of techniques to generate a sized output.

In some embodiments at Step 510 the method may utilize one or more subtractive and/or additive manufacturing systems for manufacturing the appliance for the user 102. In some embodiments the method may send the fitted output data generated at Step 508 to appliance generation device 204. Appliance generation device 204 may be configured as a 3D printer capable of printing the appliance from the fitted output data. In some embodiments the appliance generation device 204 may be configured as a subtractive manufacturing device equipped with a system for removing material from an appliance. Once generated the appliance may be retrieved by the user 102 from port 112.

FIG. 6 is a flowchart illustrating a method of training an artificial intelligence algorithm according to one embodiment of the disclosure. The method may utilize a large dataset of scans with manually annotated points for the training. The points may be from anatomical regions of a foot 110 such as for example a first metatarsal region, a third metatarsal region, a fifth metatarsal region, a navicular region, a mid-lateral arch region, a mid-heel region, a left heel region, a right heel region, and a top heel region.

In some embodiments the method at Step 602 generates uniform data for training the artificial neural network. The method may utilize one or more algorithms to pad or remove data points to normalize data sets and ensure consistency across samples.

In some embodiments at Step 604 one or more artificial neural network (ANN) algorithms are selected for training. ANN selection is dependent upon the characteristics of the training data including layers for convolution, fully connected operations, and pooling. In some embodiments the method at Step 604 may select an ANN having architecture including an input layer, a convolutional layer having 3 or more layers, a global max pooling function, and an output layer. In some embodiments the method at Step 604 may select the ANN architecture in Table 1 for training.

TABLE 1

| | |
|---|---|
| Input Layer | 10,000 points randomly sampled from the scan. (Scan |
| | data having X, Y, Z, components) |
| Convolutional | 1) First Layer (64 filters + ReLU activation function) |
| Layers | 2) Second Layer (128 filters + ReLU activation function) |
| | 3) Third Layer (256 filters + ReLu activation function) |
| Global Max | Data reduced to 256-dimensional vector |
| Pooling | (Capture important features through slide reduction) |
| Output Layers | 1) First dense layer (fully connected layers reduces 256 |
| | features to 128) |
| | 2) ReLU activation function |
| | 3) Second dense layer (30 Values final output 10 points) |

In some embodiments the architecture of the ANN may be configured with an input layer sampling fewer than 10,000 points, and may be 0 to 2,000 points, 2,000 to 4,000, 4,000 to 6,000, 6,000 to 8,000, and/or 8,000 to 10,000 points or any number of sampling points within this range. In some embodiments the architecture of the ANN may be configured with an input layer sampling more than 10,000 points, and may be 10,000 to 12,000 points, 12,000 to 14,000, 14,000 to 16,000, 16,000 to 18,000 and 18,000 to 20,000 points or any number of sampling points within this range or above. The input layer sampling ranges are not limited to the ranges described above, and may be expanded as advances in computational capabilities allow for larger ranges.

In some embodiments the convolutional layer of the ANN in Table 1 may be configured with three layers having a number of filters and a ReLU activation function. The number of filters in the first layer of the convolution layer may be configured as any one of 64, 128, 256, 512, 1024, or higher than 1024. The number of filters in the second layer may be 128, 256, 512, 1024, or higher than 1024. The number of filters in the third layer may be 256, 512, 1024, or higher than 1024. It should be noted that the number of layers in the convolution layer is not limited to three layers and may be up to 1000 layers or more. In some embodiments the ANN architecture may be configured with a plurality of convolution layers with low number of filters, for example the ANN may be configured with 50, 100, 150, 200, 250, 300 layers having a small number of filters for example 2, 4, 8, 16, and 32.

In some embodiments the ReLU activation function (rectified linear activation function) may be configured as a piecewise linear function that may output an input directly if it is positive, otherwise, it may output zero. In some embodiments the ANN may be configured to utilize alternative activation functions such as sigmoid and hyperbolic functions or any combination thereof.

In some embodiments the ANN in Table 1 may be configured with a global max pooling function that reduces the data to a single dimensional vector of any of 64, 128, 256, 512, 1024, 2048 or larger. The global max pooling function captures the most important features from the data while reducing computational complexity by reducing the number of parameters in the data. In some embodiments the ANN in Table 1 may be configured with an average max pooling function or any combination of pooling functions to capture features in the data. In some embodiments a global max pooling function may be configured to reduce the data to a multi dimensional vector of 65, 128, 256, 512, 1024, 2048 or larger.

In some embodiments the ANN in Table 1 may be configured with an output layer. The output layer may be configured with a first dense layer that is fully connected that reduces the 256 vector layer to 128. In some embodiment the ANN may be configured to apply a ReLU activation function to the first layer output. In some embodiments the output layer may be configured with a second dense output layer that produces 30 values that define the 10 points of the anatomical region. It should be noted that the output layer is not limited to two layers and may be configured with up to 100 layers. The size of the output layer may be 512, 1024, 2048 or larger and is only limited to computation capacity. It should be noted that in some embodiments the ANN may be configured with a ReLU activation function at every convolutional layer and output layer. In some embodiments the ANN may be configured with fewer ReLU activation functions in the convolutional layers and the output layer.

In some embodiments the method at Step 606 may be configured to train the ANN in Table 1 on manually curated data sets for 100 epochs. The method at Step 606 is not limited to training the ANN in Table 1 for 100 epochs, the training paradigm may include 200, 300, 400, 500, 600, 700, 800, 1000 epochs or any number of epochs within this range.

FIG. 7 shows two perspective views of a 3D scan of a user's foot. FIG. 7A is a perspective view of a transverse region of the 3D scan of the user 102 foot 110. FIG. 7B is a perspective view of sagittal region of the 3D scan of the user 102 foot 110. The following describes one embodiment in which a 3D foot scan 700 of a user 102 foot 110 may be generated in the appliance generator 100, FIG. 7 illustrates two perspective views of the 3D foot scan 700 along with surface output generated at Step 504 and the sized output generated at Step 506. In some embodiments as illustrated in FIG. 7A the system and methods of the appliance generator 100 determines points within the scan utilizing the trained ANN (not shown). In some embodiments the appliance generator 100 utilizing the points determined by the ANN generates a surface output 702 and boundary 704 utilizing one or more algorithms. This surface output 702 and boundary 704 (light gray line) may be visualized (as shown in FIGS. 7A and B) as a mesh plot and/or point cloud data having X, Y, and Z components.

In some embodiments and as illustrated as a perspective view of sagittal region of the 3D scan of the user 102 foot 110 in FIG. 7B a sized output 706 may be generated utilizing one or more algorithms. In some embodiments the system and methods utilize the data from the 3D foot scan 700 to determine an optimization of the surface output 702 and the boundary 704 to generate a fitted output 706 surface and boundary (black line). In some embodiments further steps may be carried out such as for example the fitted output method at Step 510 prior to generating the appliance with the appliance generator 100.

Although the description makes reference to systems and methods that utilize an artificial neural network (ANN), this term is used broadly to describe the features of any artificial intelligence algorithm defined by an artificial intelligence architecture including machine learning, generative adversarial network, convolution neural networks, feed forward recursive neural network algorithm, a naïve bayes classifier, decision tree, logistic regression, random forest, support vector machine, k-nearest neighbor, linear regression, lasso, k-mean clustering, multivariate regression, multiple regression algorithm, fuzzy c-means algorithm, expectation maximization algorithm, hierarchical clustering algorithm or any combination thereof the architectures may be used interchangeably or in combination.

Although some of the drawings illustrate a number of operations in a particular order, operations which are not order dependent may be reordered and other operations may be combined or removed altogether. While some reordering or other groupings are specifically mentioned, others will be apparent to those of ordinary skill in the art and so do not present an exhaustive list of alternatives. Moreover, it should be recognized that both the ordered and operations and non-ordered operations could be implemented in hardware, firmware, software or any combination thereof.

The description and drawings are illustrative and are not to be construed as limiting. The description and drawings are described to provide a thorough understanding of the disclosure. However, in certain instances, well-known or conventional details are not described to avoid obscuring the disclosure in the description. References to one or an embodiment in the present disclosure are not necessarily references to the same embodiment; and, such references mean at least one. Reference in this specification to “one embodiment” or “an embodiment” means that a referenced feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described, which may be requirements for some embodiments but not for other embodiments.

With the foregoing description, the disclosure herein has described the subject matter of the following numbered clauses:

Clause 1. A method of generating an appliance for a body part including determining, by a computer with a trained artificial neural network algorithm, at least one point from data related to the body part, generating, by the computer, with a surface generation algorithm, a surface output based on the at least one point, generating, by the computer, with the surface output and the data related to the body part, a sized output, extending, by the computer, the at least one point along a surface of the sized output, to generate a fitted output, generating, based on the fitted output, the appliance for the body part with an appliance generation device.

Clause 2. The method of clause 1, the trained artificial neural network algorithm determining at least seven points from the data related to a foot.

Clause 3. The method of clause 2, the at least seven points determined by the trained artificial neural network algorithm from a first metatarsal region, a fifth metatarsal region, a base of a 5th metatarsal region, a navicular point region, a left heel point region, a right heel point region, and a dorsal heel region of the foot.

Clause 4. The method of clause 1, training the artificial neural network algorithm including generating, by preprocessing training data and related labeled data with a computer, uniform data; and training, by the computer, an artificial neural network on the uniform data with a selected training algorithm to generate the trained artificial neural network, the selected training algorithm has a backpropagation algorithm and a gradient descent algorithm.

Clause 5. The method of clause 1, the fitted output generating by a subtractive appliance generation device.

Clause 6. The method of clause 1, the fitted output generating by an additive appliance generation device.

Clause 7. The method of clause 1, the trained artificial neural network algorithm a generative adversarial algorithm.

Clause 8. A system for generating an appliance for a body part including determine, by a computer with a trained artificial neural network algorithm, at least one point from data related to a body part, generate, by the computer, with a surface generation algorithm, a surface output based on the at least one point, generate, by the computer, with the surface output and the data related to the body part, a sized output, extending, by the computer, the at least one point along a surface of the sized output, to generate a fitted output, generate, based on the fitted output, an appliance for the body part with the appliance generation device.

Clause 9. The system of clause 8, the trained artificial neural network algorithm determines at least seven points from the data related to a foot.

Clause 10. The system of clause 9, the at least seven points determined by the trained artificial neural network algorithm from a first metatarsal region, a fifth metatarsal region, a base of a 5^{th} metatarsal region, a navicular point region, a left heel point region, a right heel point region, and a dorsal heel region of the foot.

Clause 11. The system of clause 8, train the artificial neural network algorithm including generate, by preprocessing training data and related labeled data with the computer, uniform data, the training data and the labeled data related to a body part; train, by the computer, an ANN on the uniform data with a selected training algorithm to generate the trained ANN, the selected training algorithm has a backpropagation algorithm and a gradient descent algorithm.

Clause 12. The system of clause 8, the fitted output generated by a subtractive appliance generation device.

Clause 13. The system of clause 8, the fitted output generated by an additive appliance generation device.

Clause 14. The system of clause 8, the trained artificial neural network algorithm a generative adversarial algorithm.

Clause 15. A non-transitory computer-readable medium having computer-executable instructions for a method having the steps of: determining, by a computer with a trained artificial neural network algorithm, at least one point from data related to a body part, generating, by the computer with a surface generation algorithm, a surface output based on the at least one point, generating, by the computer, with the surface output and the data related to the body part, a sized output, extending, by the computer, the at least one point along a surface of the sized output, a fitted output, generating, based on the fitted output, the appliance for the body part with an appliance generation device.

Clause 16. The non-transitory computer-readable medium of clause 15, the trained artificial neural network algorithm determining at least seven points from the data related to a foot.

Clause 17. The non-transitory computer-readable medium of clause 16, the at least seven points determined by the trained artificial neural network algorithm from a first metatarsal region, a fifth metatarsal region, a base of a 5th metatarsal region, a navicular point region, a left heel point region, a right heel point region, and a dorsal heel region of the foot.

Clause 18. The non-transitory computer-readable medium of clause 15, the fitted output generating by a subtractive appliance generation device.

Clause 19. The non-transitory computer-readable medium of clause 15, the fitted output generating by an additive appliance generation device.

Clause 20. The non-transitory computer-readable medium of clause 15, training the artificial neural network algorithm including generating, by preprocessing training data and related labeled data with a computer, uniform data, and training, by a computer, an artificial neural network on the uniform data with a selected training algorithm to generate the trained artificial neural network, the selected training algorithm has a backpropagation algorithm and a gradient descent algorithm.

In the foregoing specification, the disclosure has been described with reference to specific exemplary embodiments thereof. It will be evident that various modifications may be made thereto without departing from the broader spirit and scope as set forth in the following claims. The specification and drawings are, accordingly, to be regarded in an illustrative sense rather than a restrictive sense.

What is claimed is:

1. A method of generating an appliance for a body part comprising:
determining, by a computer with a trained artificial neural network algorithm, at least one point from data related to the body part;
generating, by the computer, with a surface generation algorithm, a surface output based on the at least one point;
generating, by the computer, with the surface output and the data related to the body part, a sized output;
extending, by the computer, the at least one point along a surface of the sized output, to generate a fitted output; and
generating, based on the fitted output, the appliance for the body part with an appliance generation device.

2. The method of claim 1, the trained artificial neural network algorithm determining at least seven points from the data related to a foot.

3. The method of claim 2, the at least seven points determined by the trained artificial neural network algorithm from a first metatarsal region, a fifth metatarsal region, a base of a 5^{th} metatarsal region, a navicular point region, a left heel point region, a right heel point region, and a dorsal heel region of the foot.

4. The method of claim 1, training the artificial neural network algorithm comprising: generating, by preprocessing training data and related labeled data with the computer, uniform data; and
training, by the computer, an artificial neural network algorithm on the uniform data with a selected training algorithm to generate the trained artificial neural network algorithm, the selected training algorithm has a backpropagation algorithm and a gradient descent algorithm.

5. The method of claim 1, the fitted output generating by a subtractive appliance generation device.

6. The method of claim 1, the fitted output generating by an additive appliance generation device.

7. The method of claim 1, the trained artificial neural network algorithm a generative adversarial algorithm.

8. A system for generating an appliance for a body part comprising;
determine, by a computer with a trained artificial neural network algorithm, at least one point from data related to the body part;
generate, by the computer, with a surface generation algorithm, a surface output based on the at least one point;
generate, by the computer, with the surface output and the data related to the body part, a sized output;
extending, by the computer, the at least one point along a surface of the sized output, to generate a fitted output; and
generate, based on the fitted output, the appliance for the body part with an appliance generation device.

9. The system of claim 8, the trained artificial neural network algorithm determines at least seven points from the data related to a foot.

10. The system of claim 9, the at least seven points determined by the trained artificial neural network algorithm from a first metatarsal region, a fifth metatarsal region, a base of a 5^{th} metatarsal region, a navicular point region, a left heel point region, a right heel point region, and a dorsal heel region of the foot.

11. The system of claim 8, train the artificial neural network algorithm comprising:
generate, by preprocessing training data and related labeled data with the computer, uniform data; and
train, by the computer, an artificial neural network algorithm on the uniform data with a selected training algorithm to generate the trained artificial neural network algorithm, the selected training algorithm has a backpropagation algorithm and a gradient descent algorithm.

12. The system of claim 8, the fitted output generated by a subtractive appliance generation device.

13. The system of claim 8, the fitted output generated by an additive appliance generation device.

14. The system of claim 8, the trained artificial neural network algorithm a generative adversarial algorithm.

15. A non-transitory computer-readable medium having computer-executable instructions for a method comprising the steps of:
determining, by a computer with a trained artificial neural network algorithm, at least one point from data related to a body part;
generating, by the computer with a surface generation algorithm, a surface output based on the at least one point;
generating, by the computer, with the surface output and the data related to the body part, a sized output;
extending, by the computer, the at least one point along a surface of the sized output, a fitted output; and
generating, based on the fitted output, an appliance for the body part with an appliance generation device.

16. The non-transitory computer-readable medium of claim 15, the trained artificial neural network algorithm determining at least seven points from the data related to a foot.

17. The non-transitory computer-readable medium of claim 16, the at least seven points determined by the trained artificial neural network algorithm from a first metatarsal region, a fifth metatarsal region, a base of a 5^{th} metatarsal region, a navicular point region, a left heel point region, a right heel point region, and a dorsal heel region of the foot.

18. The non-transitory computer-readable medium of claim 15, the fitted output generating by a subtractive appliance generation device.

19. The non-transitory computer-readable medium of claim 15, the fitted output generating by an additive appliance generation device.

20. The non-transitory computer-readable medium of claim 15, training the artificial neural network algorithm comprising: generating, by preprocessing training data and related labeled data with the computer, uniform data; and
training, by the computer, an ANN on the uniform data with a selected training algorithm to generate a trained ANN, the selected training algorithm has a backpropagation algorithm and a gradient descent algorithm.
